(19) United States
(12) Patent Application Publication
(10) Pub. No.: US 2026/0248404 A1
(43) Pub. Date: Aug. 27, 2026

(54) METHOD AND APPARATUS FOR ANALYZING SLEEP AND HEALTH STATUS USING RADAR

(71) Applicant: SEWON INTELLIGENCE Co., Ltd, Seoul (KR)

(72) Inventors: Se-jin PARK, Anseong-si (KR); Seung-hee HONG, Sejong-si (KR)

(73) Assignee: SEWON INTELLIGENCE Co., Ltd, Seoul (KR)

(21) Appl. No.: 19/061,080

(22) Filed: Feb. 24, 2025

(51) Int. Cl. A61B 5/05; A61B 5/00; A61B 5/0205; A61B 5/024; A61B 5/08; A61B 5/11; G01S 7/41; G16H 50/30

(52) U.S. Cl. CPC A61B 5/05; A61B 5/0022; A61B 5/0205; A61B 5/4815; A61B 5/725; G01S 7/415; G16H 50/30; A61B 5/02444; A61B 5/0816; A61B 5/1126

(57) ABSTRACT

Disclosed is a method and apparatus for analyzing sleep and health status using radar. In an aspect of the present disclosure, an apparatus for analyzing sleep and health status using radar according to at least one of the various embodiments of the present disclosure may include a memory; a radar sensor for sensing movement data during sleeping of a user; and a processor configured to analyze the sleep and health status by obtaining data sensed by the radar sensor, wherein the processor may be configured to convert the data sensed by the radar sensor into a frequency domain and obtain respiratory activity waveform and heartbeat waveform data.

### BACKGROUND

### 1. Technical Field

The present disclosure relates to a method and apparatus for analyzing sleep and health status using radar.

### 2. Description of Related Art

Humans spend a lot of time in a sleeping state. The amount and quality of sleep varies depending on the user's health condition, and conversely, the amount and quality of sleep can affect the recovery of the user's health condition.

For this reason, there are attempts to analyze and improve the amount and quality of sleep and health status, but in the past, the user's sleep and health status are identified through a bed or externally mounted sensors, but there is a problem that the accuracy is low due to a lot of noise.

### SUMMARY

The purpose of the embodiment disclosed in the present disclosure is to provide a method and apparatus for analyzing sleep and health status of a user using radar built into a topper.

Technical problems of the inventive concept are not limited to the technical problems mentioned above, and other technical problems not mentioned will be clearly understood by those skilled in the art from the following description.

In an aspect of the present disclosure, an apparatus for analyzing sleep and health status using radar may include a memory; a radar sensor for sensing movement data during sleeping of a user; and a processor configured to analyze the sleep and health status by obtaining data sensed by the radar sensor, wherein the processor may be configured to convert the data sensed by the radar sensor into a frequency domain and obtain respiratory activity waveform and heartbeat waveform data.

According to at least one of the embodiments of the present disclosure, the processor may be configured to filter the data sensed by the radar sensor converted into the frequency domain and obtain the respiratory activity waveform and heartbeat waveform data.

According to at least one of the embodiments of the present disclosure, the filtering may be performed by a band-pass filter for a frequency domain corresponding to the respiratory activity waveform and heartbeat waveform data.

According to at least one of the embodiments of the present disclosure, the processor may be configured to read out reference data for comparison with the respiratory activity waveform and heartbeat waveform data obtained from the memory.

According to at least one of the embodiments of the present disclosure, the reference data may include at least one of respiratory activity waveform and heartbeat waveform of the user for a predetermined period of time, respiratory activity waveform and heartbeat waveform of the user when the user is healthy and when the user is not healthy, respiratory activity waveform and heartbeat waveform of the user by sleep start time, respiratory activity waveform and heartbeat waveform of the user by total sleep time, respiratory activity waveform and heartbeat waveform of the user by day of a week, respiratory activity waveform and heartbeat waveform of the user by weather during sleep, respiratory activity waveform and heartbeat waveform of the user by a sleep position, or respiratory activity waveform and heartbeat waveform of the user by a sleep posture.

According to at least one of the embodiments of the present disclosure, the reference data may further include at least one of respiratory activity waveform and heartbeat waveform received from an external medical institution database server, or respiratory activity waveform and heartbeat waveform of a healthy person and a person suffering from a given disease from the external medical institution database server.

According to at least one of the embodiments of the present disclosure, a priority and a weight of each of the reference data may be different.

According to at least one of the embodiments of the present disclosure, the priority and the weight of each of the reference data may be different depending on a time of the sleep and health status analysis.

According to at least one of the embodiments of the present disclosure, the processor may be configured to control to generate and transmit guide data including recommended data and content based on the sleep and health status analysis result.

In another aspect of the present disclosure, a method for analyzing sleep and health status using radar may include storing reference data; obtaining movement data of a user during sleeping using the radar; converting the obtained movement data of the user during sleeping into a frequency domain and obtaining respiratory activity waveform and heartbeat waveform data; analyzing the obtained respiratory and heartbeat waveform data by comparing the data with the stored reference data; and generating and transmitting guide data including recommended content based on the analysis result.

Other specific details of the present disclosure are included in the detailed description and drawings.

### BRIEF DESCRIPTION OF THE FIGURES

FIG. 1 is a diagram for describing a system according to an embodiment of the present disclosure.

FIG. 2 is a diagram for describing a processor in a topper of FIG. 1.

FIGS. 3A-3B and 4A-4B are diagrams illustrating an arrangement of the radar in the topper.

FIGS. 5 and 6 are flowcharts illustrating a method for analyzing sleep and health status of a user using radar according to an embodiment of the present disclosure.

FIGS. 7A-7B are diagram illustrated for describing an example of a sleep and health status analysis guide according to an embodiment of the present disclosure.

### DETAILED DESCRIPTION

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

The operating principle and embodiments of the present disclosure are described below with reference to the attached drawings.

In this specification, the term ‘apparatus according to the present disclosure’ includes all of various devices that can perform computational processing and provide results to the user. For example, the device may include all of a computer, a server device, and a portable terminal, or may be in the form of one of them.

Here, the computer may include, for example, a notebook, a desktop, a laptop, a tablet PC, a slate PC, and the like mounted with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

The function related to artificial intelligence according to the present disclosure operates through a processor and a memory. The processor may be composed of one or more processors. At this time, the one or more processors may be a general-purpose processor such as a CPU, an AP, a DSP (Digital Signal Processor), a graphics-only processor such as a GPU, a VPU (Vision Processing Unit), or an artificial intelligence-only processor such as an NPU. The one or more processors control input data to be processed according to a predefined operation rule or artificial intelligence model stored in the memory. Alternatively, in the case that the one or more processors are artificial intelligence-only processors, the artificial intelligence-only processor may be designed as a hardware structure specialized for processing a specific artificial intelligence model.

The predefined operation rule or artificial intelligence model may be created through learning. Here, being created through learning means that a basic artificial intelligence model is learned by using a plurality of learning data by a learning algorithm, thereby creating a predefined operation rule or artificial intelligence model set to perform a desired characteristic (or, purpose). Such learning may be performed on the device itself in which the artificial intelligence according to the present disclosure is performed, or may be performed through a separate server and/or system. Examples of learning algorithms include supervised learning, unsupervised learning, semi-supervised learning, or reinforcement learning, but are not limited to the examples described above.

The artificial intelligence model may include a plurality of neural network layers. Each of the plurality of neural network layers has a plurality of weights, and performs neural network operations through operations between the operation results of the previous layer and the plurality of weights. The plurality of weights of the plurality of neural network layers may be optimized by the learning results of the artificial intelligence model. For example, the plurality of weights may be updated so that the loss value or cost value acquired by the artificial intelligence model is reduced or minimized during the learning process. The artificial neural network may include a deep neural network (DNN), for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the examples described above.

According to an exemplary embodiment of the present disclosure, the processor may implement artificial intelligence. Artificial intelligence refers to a machine learning method based on an artificial neural network that imitates human neurons (biological neurons) to enable a machine to learn. The artificial intelligence methodology may be divided into supervised learning in which input data and output data are provided together as training data according to a learning method so that the answer (output data) to a problem (input data) is determined, unsupervised learning in which only input data is provided without output data so that the answer (output data) to a problem (input data) is not determined, and reinforcement learning in which a reward is given from an external environment whenever an action is taken in a current state (State), and learning is performed in a direction to maximize this reward. In addition, the methodology of artificial intelligence can be classified according to the architecture, which is the structure of the learning model. The architecture of widely used deep learning technology can be classified into convolutional neural network (CNN), recurrent neural network (RNN), transformer, and generative adversarial network (GAN).

The present device and system may include an artificial intelligence model. The artificial intelligence model may be one artificial intelligence model or may be implemented as multiple artificial intelligence models. The artificial intelligence model may be composed of a neural network (or artificial neural network) and may include a statistical learning algorithm that mimics the neurons of biology in machine learning and cognitive science. A neural network may mean an overall model that has problem-solving capabilities by changing the strength of the synapse connection through learning by forming a network with artificial neurons (nodes) that combine synapses. The neurons of the neural network may include a combination of weights or biases. The neural network may include one or more layers composed of one or more neurons or nodes. For example, the device may include an input layer, a hidden layer, and an output layer. The neural network constituting the device can infer a desired result (output) from an arbitrary input (input) by changing the weights of neurons through learning.

The processor may generate a neural network, train (or learn) a neural network, perform a calculation based on received input data, generate an information signal based on the result of the calculation, or retrain the neural network. The models of the neural network may include various types of models such as CNN (Convolution Neural Network) such as GoogleNet, AlexNet, VGG Network, R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restrcted Boltzman Machine), Fully Convolutional Network, LSTM (Long Short-Term Memory) Network, Classification Network, and the like, but are not limited thereto. The processor may include one or more processors for performing calculations according to the models of the neural network. For example, a neural network may include a deep neural network.

The neural network may include CNN (Convolutional Neural Network), RNN (Recurrent Neural Network), percept, multilayer perceptron, FF (Feed Forward), RBF (Radial Basis Network), DFF (Deep Feed Forward), LSTM (Long Short Term Memory), Gated Recurrent Unit (GRU), Auto Encoder (AE), Variational Auto Encoder (VAE), Denoising Auto Encoder (DAE), Sparse Auto Encoder (SAE), Markov Chain (MC), Hopfield Network (HN), Boltzmann Machine (BM), Restricted Boltzmann Machine (RBM), Depp Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine (ELM), Echo State Network (ESN), Deep Residual Network (DRN), Differentiable Neural Computer (DNC), Neural Turning Machine (NTM), Capsule Network (CN), Kohonen Network (KN), and Attention Network (AN), but not limited thereto, and it will be understood by those skilled in the art that any neural network may be included.

According to an exemplary embodiment of the present disclosure, the processor may use various artificial intelligence structures and algorithms such as CNN (Convolution Neural Network), R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restricted Boltzmann Machine), Fully Convolutional Network, LSTM (Long Short-Term Memory) Network, Classification Network, Generative Modeling, eXplainable AI, Continual AI, Representation Learning, and AI for Material Design such as GoogleNet, AlexNet, VGG Network, BERT, SP-BERT, MRC/QA, Text Analysis, Dialog System, GPT-3, and GPT-4 for natural language processing, Visual Analytics, Visual Understanding, Video Synthesis for vision processing, Anomaly Detection, Prediction, Time-Series Forecasting, Optimization, and Recommendation for algorithms ResNet for data intelligence, but not limited thereto. Hereinafter, the embodiment of the present disclosure will be described in detail.

Hereinafter, a method and apparatus for analyzing sleep and health status using radar according to the present disclosure will be described in this specification.

The apparatus for analyzing sleep and health status using radar according to the present disclosure in this specification may include various devices that may perform computational processing and provide results to a client. For example, the apparatus according to the present disclosure may include at least one computer or computing device, a server device, a terminal, and the like, or may be in the form of one of them.

In the above description, the computer may include, for example, a notebook, desktop, laptop, tablet PC, slate PC, and the like provided with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

Hereinafter, the embodiment of the present disclosure will be described in detail with reference to the attached drawings.

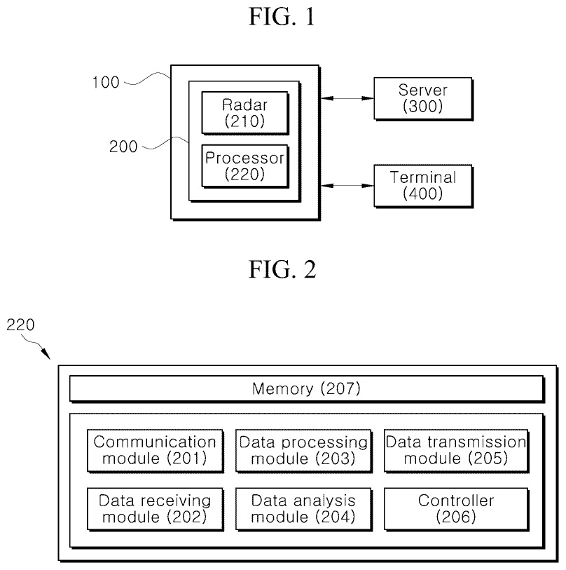

FIG. 1 is a diagram for describing a system according to an embodiment of the present disclosure.

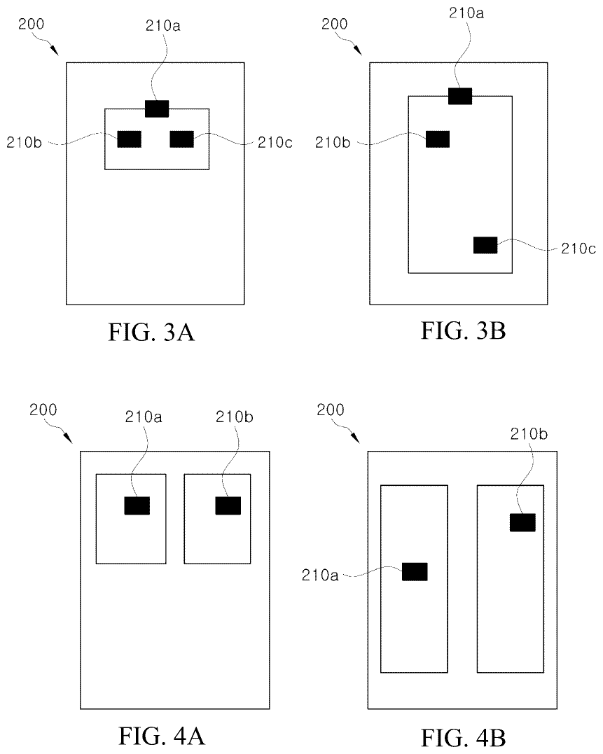

FIG. 2 is a diagram for describing a processor in a topper of FIG. 1.

Referring to FIG. 1, a sleep and health status analysis system 1 using radar may include a bed 100, a topper 200, a server 300, and a terminal 400.

The topper 200 that may be placed on the bed 100 may include a radar 210 and a processor 220.

The radar 210 may include at least one radar sensor or radar sensor module. The radar 210 may sense a movement of a user and collect data on the user's breathing, heartbeat, and the like.

The processor 220 may receive and process data for analyzing the sleep and health status of the user collected by the radar 210. The result of the processing may be transmitted to the server 300 or/and the terminal 400.

Referring to FIG. 2, the processor 220 may include the following components to process data collected by the radar 210.

The processor 220 may include a communication module 201, a data receiving module 202, a data processing module 203, a data analysis module 204, a data transmission module 205, a controller 206, and the like.

The communication module 201 may support a wired/wireless communication protocol for communication with the radar 210 and an external device. Here, the external device may include the server 300 and/or the terminal 400. Meanwhile, the external device may include various devices that store or retain data related to status analysis, and the like in relation to the present disclosure in addition to the above configuration. The wired/wireless communication protocol may include, but is not limited to, Bluetooth, BLE, Zigbee, Wi-Fi, 3G, 4G, 5G, and the like.

The data receiving module 202 may receive data on the user's movement sensed by the radar 210 connected through the communication module 201.

The controller 206 processes only meaningful data related to the user's health status, including the user's breathing and heart status during sleeping, from the user's movement sensing data during sleeping according to the present disclosure, and removes other noises, collects various data unrelated to the user's movement during sleeping, stores them in advance in the memory 207, and may use them for filtering thereafter.

The data processing module 203 processes the user's movement data collected through the radar 210 received by the data receiving module 202, filters only the data related to the user's breathing and health status during sleeping, and processes the filtered data so that it may be analyzed separately. For convenience, the data related to the health status will be described below using, for example, heart status data as an example.

When the data processing module 203 receives the user's movement data during sleeping from the data receiving module 202, it may call the noise data from the memory 207 and compare it to remove the noise. The data from which the noise is removed may be converted into a frequency domain by processing FFT (Fast Fourier Transform). The data processing module 203 separates the user's movement data during sleeping, which has been converted into a frequency domain by processing FFT, into frequency domain modules, and individually processes the data of each separated region to generate data for analyzing breathing during sleeping and data for analyzing health status. Each analysis data generated in this manner may be directly transferred to the data analysis module 204 or transferred to the data analysis module 204 after processing IFFT (Inverse FFT).

In the above description, when the data processing module 203 receives the user's movement data during sleeping from the data receiving module 202, it first performs FFT processing without filtering it and converts it into a frequency domain, and filters the data converted into the frequency domain to extract only the respiratory activity waveform and heartbeat waveform during sleeping. The filtering may be, for example, one of a low-pass filter, a high-pass filter, or a band-pass filter depending on the noise. The subsequent operation may refer to the above-mentioned content.

The data analysis module 204 may analyze the user's quality of sleep and health status by calling and comparing the respiratory activity waveform and heartbeat waveform extracted by the data processing module 203 with the corresponding respiratory activity waveform and heartbeat waveform from the memory 207.

In the data analysis process, the following reference data for analysis may be stored in advance in the memory 207.

The reference data may include, for example, at least one of the following: the respiratory activity waveform and heartbeat waveform of the user during a given period of time; the respiratory activity waveform and heartbeat waveform of the user when the user is healthy or not healthy; the respiratory activity waveform and heartbeat waveform at each sleeping start time of the user; the respiratory activity waveform and heartbeat waveform of the user for each of the entire sleep time; the respiratory activity waveform and heartbeat waveform of the user by day of the week; the respiratory activity waveform and heartbeat waveform of the user by weather during sleeping; the respiratory activity waveform and heartbeat waveform of the user by sleeping position; the respiratory activity waveform and heartbeat waveform of the user according to the sleeping posture; the respiratory activity waveform and heartbeat waveform received from an external medical institution database server in each of the above cases; the respiratory activity waveform and heartbeat waveform of a generally healthy person and a person suffering from a given disease from an external medical institution database server; and the like.

The reference data may be given different priorities or weights during sleep analysis. The priorities or weights may also be different depending on the analysis time. For example, in the case that today is Wednesday and it is rainy, the user's sleep start time is 12 midnight, and the total sleep time is 6 hours, then the reference data corresponding to Wednesday, rain, 12 midnight, and 6 hours may be given higher priority or weight. Accordingly, when applying multiple reference data simultaneously or sequentially to analyze the respiratory activity waveform and heartbeat waveform of the user during sleep, the user's health status may be analyzed with reference to the above-mentioned priorities or weights, and the guide including recommended data or content may be provided differently accordingly.

The memory 207 may process and store the critical respiratory activity waveform and heartbeat waveform for health and health abnormalities in advance. Therefore, when the data analysis module 204 first obtains the respiratory activity waveform and heartbeat waveform information of the user during sleeping, it may determine whether to perform secondary processing on the respiratory activity waveform and heartbeat waveform information obtained by applying the critical respiratory activity waveform and heartbeat waveform. For example, when the data analysis module 204 determines that there is a problem and secondary processing is necessary based on the first critical respiratory activity waveform and heartbeat waveform information of the user during sleeping, it may set this as additional health status determination candidate data.

When the data analysis module 204 sets the additional health status determination candidate data, it may skip the primary processing process and perform the secondary processing process directly on the subsequent data. The secondary processing process may refer to comparing and analyzing the respiratory activity waveform and heartbeat waveform more accurately by calling and comparing at least one candidate data from the memory 207 to determine the user's health status. Afterwards, in the case that the data analysis module 204 determines that there is no problem with the health status for a predetermined number of times for example, three times or more, the data analysis module 204 may switch back to the primary processing priority application procedure.

When the data analysis module 204 first performs the secondary processing process, the health status may be determined by applying only a predetermined number of the highest priority reference data according to the priority or weight, but in the case that it continues to determine that the health status is abnormal, it may use additional reference data to determine the accurate health status.

In the case that the data analysis module 204 determines that the user's sleeping respiratory activity waveform and heartbeat waveform are continuously determined to be in a healthy state for a predetermined time or a predetermined number of times based on the analysis result, the respiratory activity waveform and heartbeat waveform for a predetermined time or a predetermined number of times may be ignored and not processed. For example, when analyzing a waveform in a unit of time, the data analysis module 204 may skip the waveforms in the next hour the next hour and process the waveforms in the next hour the next hour and two hours after the next hour.

In the above description, when analyzing a waveform in a unit of time, in the case that the analysis result from the waveform for the first 10 minutes is determined to be good health, the analysis term may be increased. For example, the analysis term may be adjusted from analyzing a waveform in a unit of 1 minute to a unit of 5 minutes, 10 minutes, 20 minutes, 30 minutes, and the like. Or, in the case that the waveform analysis result for the first 10 minutes is good, the waveforms for the remaining 50 minutes may not be analyzed.

The data transmission module 205 may generate a signal including recommendation information according to the analysis result and transmit the generated signal.

Here, the recommended information may include, for example, recommended music content, recommended video content, and the like.

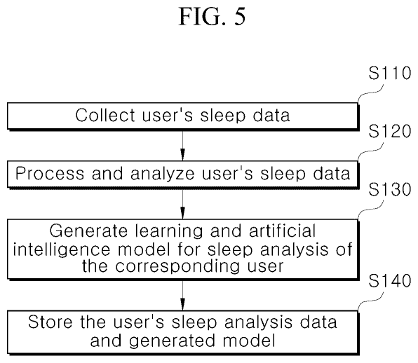

FIGS. 3A-3B and 4A-4B are diagrams illustrating an arrangement of the radar 210 in the topper 200. This may be for sensing more accurate or less noisy user sleeping movement data through the radar 210.

In FIGS. 3A-3B and 4A-4B, the square box is implemented in the form of a rail, so that the radar sensor moves along the rail and its position may be moved according to the user's movement during sleep. This may be determined, for example, using the intensity of the sensing signal. Accordingly, in the case that the intensity of the received signal of the radar 210 is below a threshold, the processor 220 may control the movement of the radar 210 to move along the rail to a point where the intensity of the received signal is above the threshold. Alternatively, the square box in FIGS. 3 and 4 may represent an area where the radar 210 may be placed.

Referring to FIGS. 3A and 3B, a predetermined space may be formed in the topper 200 where the radar 210 is positioned or may be placed. In the case of the rail form, the radar 210a may move along the rail or may be placed at a position close to the user's lungs or heart, which is estimated based on the user's movement data during sleep.

In the case that FIGS. 3A and 3B describe the position of the radar within the topper for a single user, FIGS. 4A and 4B describe the position of the radar within the topper for a dual user.

In FIGS. 4A and 4B, the same as FIGS. 3A and 3B are applied, but each independent radar sensor may be placed within each independent area.

In the case of FIGS. 4A and 4B, the data processing module 203 may separate independent respiratory activity waveforms and heartbeat waveforms for each user, and transmit the respiratory activity waveforms and heartbeat waveforms for each user to the data analysis module 204 by distinguishing them.

To this end, the controller 206 may operate each radar 210a or 210b simultaneously, but may control them to operate independently or alternately at different times to increase accuracy and reduce processing time. In addition, in this case, at least one of the data receiving module 202, the data processing module 203, and the data analysis module 204 may be included in the processor 220 in multiple forms.

The functions performed by at least one of the components of the processor 220 of FIG. 2 described above may also be performed by the component of the server 300 or the terminal 400.

According to the embodiment, the topper 200 collects data through the radar 210, and the processor 220 transmits a signal including the data raw data collected by the radar 210 to at least one of the server 300 or the terminal 400, and the data may be directly processed and analyzed in the corresponding configuration, and the analysis result may be returned to the terminal 400 or the processor 220 of the topper. The results analyzed in this way may be used later to control the operation of the radar 210 of the topper 200.

In FIG. 2, at least one of the data processing module 203, the data analysis module 204, and the control module 206 may include an artificial intelligence engine AI engine not shown. The AI engine may learn using sleep data respiratory activity waveform, heartbeat waveform, etc. collected from a user or an external device as a training data set, and may generate a learning model. The learning model generated in this way may function to replace some of the operations of the data processing module 203 and/or the data analysis module 204 described above.

The server 300 may be positioned in the home or remotely, and may communicate with components of the topper 200 based on an intranet or the Internet to exchange data.

The server 300 may replace all or at least part of the functions of the processor 220 included in the topper 200.

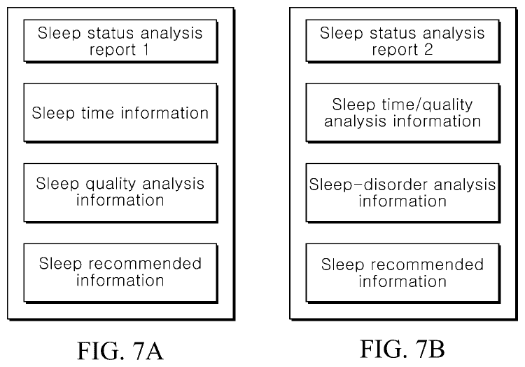

The terminal 400 may communicate with the processor 220 of the topper 200 and/or the server 300, and may receive and output the results of analyzing the data on sleep and health status collected through the radar 210 of the topper 200. Here, the results may also include guide data such as a health recommendation according to the analysis, such as FIGS. 7A and 7B, for example.

The terminal 400 may be a terminal held by the user or a terminal of a user registered in the topper 200 or the server 300. The terminal 400 may be a fixed terminal such as a PC or a TV, or a mobile terminal such as a smartphone, a tablet PC, or a laptop.

The terminal 400 may also manually control the radar 210 in the topper to collect data for analyzing the user's sleep and health status. At this time, a signal regarding the control may be transmitted through the server 300 or directly to the topper 200.

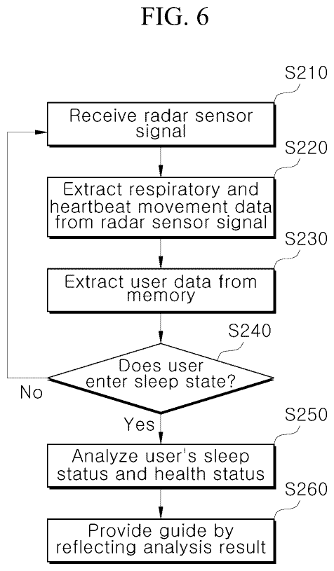

FIGS. 5 and 6 are flowcharts illustrating a method for analyzing sleep and health status of a user using radar according to an embodiment of the present disclosure.

In an electronic apparatus according to at least one of the various embodiments of the present disclosure, the method for analyzing sleep and health status of a user using radar may include storing reference data; obtaining movement data of a user during sleeping using the radar; converting the obtained movement data of the user during sleeping into a frequency domain and obtaining respiratory activity waveform and heartbeat waveform data; analyzing the obtained respiratory and heartbeat waveform data by comparing the data with the stored reference data; and generating and transmitting guide data including recommended content based on the analysis result.

Referring to FIG. 5, in operation step S110, the processor 220 may collect the user's sleep data.

In operation step S120, the processor 220 may process and analyze the user's sleep data.

In operation step S130, the processor 220 may generate a learning and artificial intelligence model for sleep analysis of the corresponding user.

In operation step S140, the processor 220 may store the user's sleep analysis data and the generated model in the memory 207.

Referring to FIG. 6, in operation step S210, the processor 220 may receive a radar sensor signal.

In operation step S220, the processor 220 may extract respiratory and heartbeat movement data, that is, the respiratory activity waveform and heartbeat waveform, from the radar sensor signal.

In operation step S230, the processor 220 may extract the data corresponding to user from the memory 207.

In operation step S240, the processor 220 may compare the two data described above to determine whether the user enters a sleep state.

In operation step S250, in the case that the determination result of operation step S240 determines that the user enters a sleep state, the processor 220 may analyze the user's sleep status and health status.

In operation step S260, the processor 220 may generate and provide a guide such as FIGS. 7A and 7B by reflecting the analysis result.

The steps of the method or algorithm described in relation to the embodiments of the present disclosure may be implemented directly in hardware, implemented as a software module executed by hardware, or implemented by a combination of these. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer readable recording medium well known in the art to which the present disclosure pertains.

Although the embodiments of the present disclosure have been described above with reference to the attached drawings, those skilled in the art will understand that the present disclosure may be implemented in other specific forms without changing the technical spirit or essential characteristics thereof. Therefore, it should be understood that the embodiments described above are exemplary in all respects and not restrictive.

According to at least one of the various embodiments of the present disclosure, by utilizing the radar disposed in the topper, there is an advantage in that more accurate analysis and response to sleep and health status may be performed.

The effects of the present disclosure are not limited to the effects mentioned above, and other effects not mentioned will be clearly understood by those skilled in the art from the description.

What is claimed is:

1. An apparatus for analyzing sleep and health status using radar, comprising:
a memory;
a radar sensor for sensing movement data during sleeping of a user; and
a processor configured to analyze the sleep and health status by obtaining data sensed by the radar sensor,
wherein the processor is configured to convert the data sensed by the radar sensor into a frequency domain and obtain respiratory activity waveform and heartbeat waveform data.

2. The apparatus according to claim 1, wherein the processor is configured to:
filter the data sensed by the radar sensor converted into the frequency domain and obtain the respiratory activity waveform and heartbeat waveform data.

3. The apparatus according to claim 2, wherein the filtering is performed by a band-pass filter for a frequency domain corresponding to the respiratory activity waveform and heartbeat waveform data.

4. The apparatus according to claim 3, wherein the processor is configured to:
read out reference data for comparison with the respiratory activity waveform and heartbeat waveform data obtained from the memory.

5. The apparatus according to claim 3, wherein the reference data includes at least one of respiratory activity waveform and heartbeat waveform of the user for a predetermined period of time, respiratory activity waveform and heartbeat waveform of the user when the user is healthy and when the user is not healthy, respiratory activity waveform and heartbeat waveform of the user by sleep start time, respiratory activity waveform and heartbeat waveform of the user by total sleep time, respiratory activity waveform and heartbeat waveform of the user by day of a week, respiratory activity waveform and heartbeat waveform of the user by weather during sleep, respiratory activity waveform and heartbeat waveform of the user by a sleep position, or respiratory activity waveform and heartbeat waveform of the user by a sleep posture.

6. The apparatus according to claim 5, wherein the reference data further includes at least one of respiratory activity waveform and heartbeat waveform received from an external medical institution database server, or respiratory activity waveform and heartbeat waveform of a healthy person and a person suffering from a given disease from the external medical institution database server.

7. The apparatus according to claim 6, wherein a priority and a weight of each of the reference data are different.

8. The apparatus according to claim 7, wherein the priority and the weight of each of the reference data are different depending on a time of the sleep and health status analysis.

9. The apparatus according to claim 8, wherein the processor is configured to:
control to generate and transmit guide data including recommended data and content based on the sleep and health status analysis result.

10. A method for analyzing sleep and health status using radar, the method performed by a processor of an electronic apparatus comprising:
storing reference data;
obtaining movement data of a user during sleeping using the radar;
converting the obtained movement data of the user during sleeping into a frequency domain and obtaining respiratory activity waveform and heartbeat waveform data;
analyzing the obtained respiratory and heartbeat waveform data by comparing the data with the stored reference data; and
generating and transmitting guide data including recommended content based on the analysis result.
